(19) United States
(12) Patent Application Publication
(10) Pub. No.: US 2014/0215736 A1
(43) Pub. Date: Aug. 7, 2014

(54) MINIMALLY INVASIVE APPARATUS AND METHOD FOR CLEANING ENDOSCOPE LENSES

(71) Applicants: Ricardo Alexander Gomez, Lighthouse Point, FL (US); Sandy Lawrence Heck, Los Angeles, CA (US)

(72) Inventors: Ricardo Alexander Gomez, Lighthouse Point, FL (US); Sandy Lawrence Heck, Los Angeles, CA (US)

(73) Assignee: NEXT WAVE SURGICAL LLC, Pompano Beach, FL (US)

(21) Appl. No.: 14/172,663

(22) Filed: Feb. 4, 2014

Related U.S. Application Data
(60) Provisional application No. 61/761,083, filed on Feb. 5, 2013.

(51) Int. Cl. A61B 1/12

(52) U.S. Cl. CPC A61B 1/122
USPC 15/104.05

(57) ABSTRACT

The disposable apparatus for cleaning a distal lens of an endoscope comprises a hollow tube having an open proximal end and an openable distal end and is sized and configured to receive an endoscope therethrough, the apparatus including a plurality of lens cleaning members disposed across an inner diameter of the hollow tube, the cleaning members each including a slit through which the distal lens passes with the lint-free material of the cleaning members wiping across the lens as it passes therethrough.

### CROSS REFERENCE TO RELATED APPLICATIONS

This application claims priority from provisional patent application Ser. No. 61/761,083 filed on 5 Feb. 2013 and entitled Minimally Invasive Apparatus and Method for Cleaning Laparoscopic Lenses, the teachings of which are incorporated herein in their entirety by reference.

### FIELD OF THE INVENTION

The present invention relates generally to devices and methods in the field of endoscopic surgery, and most particularly to manual and robotic laparoscopic surgery. More specifically, the present invention relates to an apparatus and method for cleaning a distal lens of an endoscope during a surgical procedure. In the below description, the use in cleaning a distal laparoscope is emphasized as exemplary but should not be construed as limiting in nature.

### BACKGROUND OF THE INVENTION

Endoscopic surgery has wide application in treating conditions of the human body. As an example, laparoscopic surgery, also called minimally invasive surgery (MIS), is a modern surgical technique in which operations in the abdomen are performed through small incisions (usually 0.5-1.5 cm) as opposed to the larger incisions needed in laparotomy. Laparoscopic surgery often makes use of images displayed on TV monitors to magnify the surgical elements.

Laparoscopic surgery includes operations within the abdominal or pelvic cavities. As another example, when similar techniques are used to perform surgery on the thoracic or chest cavity, the technique is referred to as thoracoscopic surgery.

The key element in laparoscopic surgery is the use of a laparoscope. There are two types: (1) a telescopic rod lens system, that is usually connected to a video camera (single chip or three chip), or (2) a digital laparoscope where the charge-coupled device is placed at the end of the laparoscope, eliminating the rod lens system. Also attached is a fiber optic cable system connected to a ‘cold’ light source (halogen or xenon), to illuminate the operative field, inserted through a 5 mm or 10 mm cannula or cannula to view the operative field.

Thus, during a laparoscopic surgical procedure, small incisions of up to half an inch are made and plastic tubes called ports are placed through these incisions. The camera and the instruments are then introduced through the ports which allow access to the inside of the patient. The camera transmits an image of the organs inside the abdomen onto a television monitor. The surgeon is not able to see directly into the patient without the traditional large incision. The video camera becomes a surgeon's eyes in laparoscopic surgery, since the surgeon uses the image from the video camera positioned inside the patient's body to perform the procedure.

The human hand performs many functions during surgery that are difficult to reproduce with laparoscopic instruments. The loss of the ability to place the hand into the abdomen during traditional laparoscopic surgery has limited the use of laparoscopy for complex abdominal surgery on the pancreas, liver, and bile duct.

Computer assisted systems have been developed to perform some more complex minimally invasive surgeries. One such system is the Da Vinci™ computer-assisted robotic system which expands a surgeon's capability to operate within the abdomen in a less invasive way during laparoscopic surgery. The Da Vinci™ computer-assisted robotic surgical system is claimed to allow greater precision and better visualization compared to standard laparoscopic surgery.

Computer assisted robotic surgery may be performed with no direct mechanical connection between the surgeon and the patient such that the surgeon is at a computer console with a three-dimensional view of the operating field and remote from the operating table and patient during surgery.

Whether surgery is completed by hand or robotically assisted, laparoscopic surgeries provide significant advantages to patients, including smaller incisions, and faster procedures. These advantages reduce the risks of infection and complications during and after surgery as well as reducing post-surgical pain and recovery time, both of which are negatively affected by large surgical incisions and long surgical procedures.

One problem with laparoscopic surgery is the need to clean the laparoscope during procedures in order for the surgeon to maintain a visible surgical field. Often, the laparoscope must be withdrawn completely from the surgical site, cleaned, and then reinserted into the cannula. Performing a cleaning in this manner is time consuming, disrupts the flow of the procedure, since the surgeon must find again the surgical site he was last in, and it is especially difficult in robotic assisted surgical procedures.

Systems exist for cleaning the laparoscope within the surgical site. However, the current systems require larger incisions, implantable cleaning devices, or specially modified cannulas. The current invention is unique in that it will function with current cannulas, is not implantable, and is the only device designed for robotic laparoscopic surgery.

For example, U.S. Pat. No. 7,311,660 (the “'660 patent”), by the present inventor, the disclosure of which is hereby incorporated by reference into the present application, discloses a sterile disposable apparatus for cleaning a medical scope.

U.S. Pat. No. 6,354,992 (the “'992 patent”), the disclosure of which is hereby incorporated by reference into the present application, discloses a multi-port cleaning apparatus having irrigation and suction functionality. Unfortunately, the apparatus of the '992 patent may require a larger than desirable incision, and may have a much higher production cost than the present invention. Also, washing the laparoscope lens without wiping the lens will not always result in the lens being cleaned completely. Hence, the apparatus of the '992 patent may still require that the laparoscope be removed from the body to completely clean the lens.

Another prior art reference, U.S. Pat. No. 5,392,766 (the “'766 patent”), the disclosure of which is hereby incorporated by reference into the present application, discloses devices for percutaneous cleaning of medical viewing scopes. However, while the device disclosed in the '766 patent provides a cleaning apparatus that reduces the need to remove the laparoscope completely from the cannula, it does not provide for a plurality of cleaning members for more effective cleaning of the scope as in the present invention.

A disposable system for cleaning a laparoscope is disclosed in United States Patent Application Publication No. 20060293559, said disclosure being incorporated herein by reference.

Finally, U.S. Pat. No. 5,225,001 (the “'001 patent”), the disclosure of which is hereby incorporated by reference into the present application, discloses a mechanical scope cleaning apparatus having a coupler for attachment to a separate cleaning solution delivery device. Thus, the device is not as simple to use as the more self-contained system of the present invention.

Unfortunately, the device disclosed in the '660 patent, while highly functional, is intended to address a different problem than the present invention. Moreover, the other prior art references disclose more complicated, more costly, or larger than desired devices. Any of these factors may increase the time of the surgical procedure or the size of a surgical incision, which can reduce the advantages of sought by performing a laparoscopic surgery in the first instance.

Thus, a need exists for a method and apparatus for cleaning a laparoscope that does not require a larger incision or removal of the laparoscope during surgery for cleaning the lens of laparoscope. A further need exists for a simple robust cleaning device.

### SUMMARY OF THE INVENTION

The present invention provides an apparatus for cleaning a distal lens of an endoscope and in an exemplary manner, of a laparoscope of the type used in modern minimally invasive manual or robotic surgeries. As discussed previously, other devices for cleaning a surgical laparoscope are known. However, most prior art devices and methods of cleaning a laparoscope suffer from one or more limitations. Most often, prior art systems require a laparoscope to be fully removed from a corresponding cannula to be cleaned. This can be both cumbersome and time consuming, especially for robotic surgical procedures. It is well understood that any increase in the time during which a patient is anesthetized or has an open surgical incision can increase the risk of a negative surgical outcome due to complications, prolonged exposure to anesthetic, infection, or other complications, especially for higher risk patients.

Additionally, many prior art systems that do not require the removal of the laparoscope for cleaning, particularly those described in the background section herein, require larger initial surgical incisions to accommodate the cleaning device or system. A larger incision not only increases the opportunity for infection in the patient, it also necessarily increases patient trauma and pain. Other systems require the use of an entire cannula to insert their cleaning system intra-abdominally, which would either result in an extra incision to place an additional cannula, or would occupy an existing cannula that could be needed for another purpose. These issues increase the time for the patient to completely heal and recover from surgery. Thus, a need exists for a method and apparatus for cleaning a laparoscope that does not require a larger incision, occupy an entire cannula, or require complete removal of the laparoscope during surgery for cleaning the lens of laparoscope.

In one exemplary embodiment, the present invention, an apparatus for cleaning a distal lens of an endoscope, with a laparoscope being exemplified, comprises a hollow tube having a longitudinal axis and an inner surface configured to slidably receive a viewing end of a laparoscope. The hollow tube further comprises an outer surface configured to be slidably received within a cannula. The proximal end of hollow tube further comprises an opening and a means for preventing the proximal end of the hollow tube from entering the cannula, preferably a surface that extends beyond the inner diameter of the cannula.

In this exemplary embodiment, the hollow tube further includes an open distal end and at least one cleaning member disposed across the longitudinal axis of said hollow tube and within the distal portion of said hollow tube. The cleaning member(s) are configured to contact and wipe at least a portion of the viewing end of an endoscope such as a laparoscope as the scope is translated through the hollow tube.

Advantageously, the present invention provides a means for cleaning such laparoscopes without the need to completely remove the laparoscope from a corresponding cannula during a surgical procedure, which can be both time consuming and cumbersome. Moreover, the present invention provides a means of cleaning a laparoscope while maintaining the same size incision.

These and other aspects, features, and advantages of the present invention will become more readily apparent from the attached drawings and the detailed description of the preferred embodiments, which follow.

### BRIEF DESCRIPTION OF THE DRAWINGS

The preferred embodiments of the invention will hereinafter be described in conjunction with the appended drawings provided to illustrate and not to limit the invention, in which:

FIG. 1 presents an external, perspective view of a cleaning apparatus according to an aspect of the present invention;

FIG. 2 presents a close-up, external, perspective view of the distal portion of the cleaning apparatus of FIG. 1;

FIG. 3A presents an enlarged exploded view of the distal portion of the cleaning apparatus and cleaning members;

FIG. 3B presents a close-up perspective view of an alternative orientation of the cleaning members;

FIG. 4 presents a longitudinal cross sectional view of the apparatus, showing it in use, and

FIG. 5 presents a sectional view of an alternative embodiment whereby flaps are used in a nested formation.

Like reference numerals refer to like parts throughout the several views of the drawings.

### DETAILED DESCRIPTION OF EXEMPLARY EMBODIMENTS

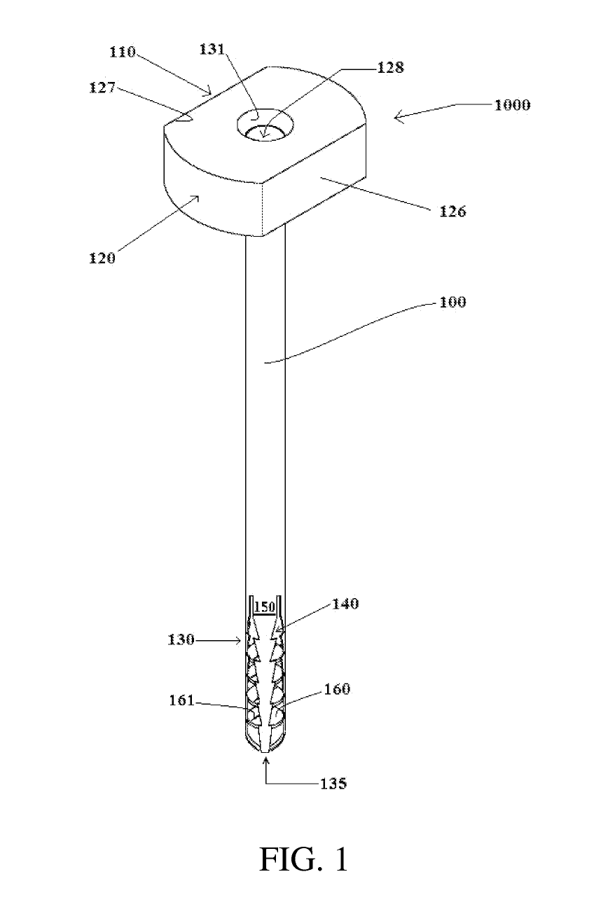

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, “proximal”, “distal”, and derivatives thereof shall relate to the invention as oriented in FIG. 1.

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

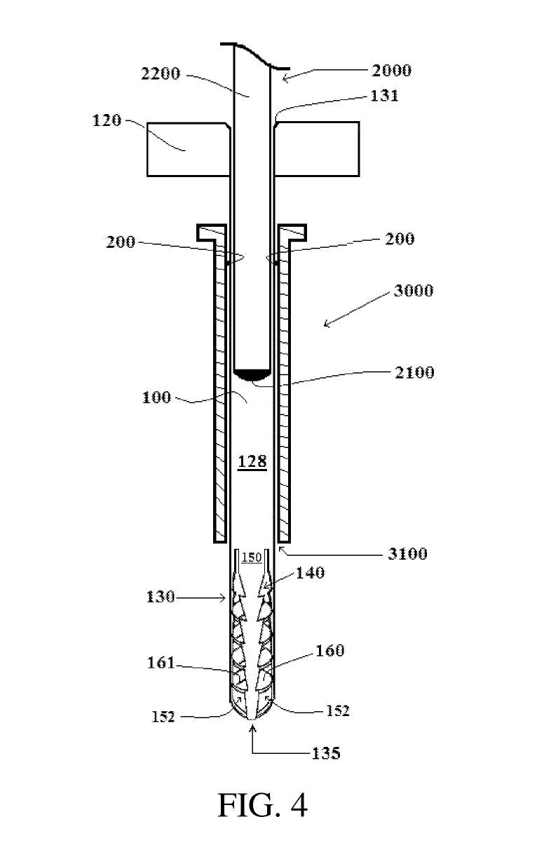

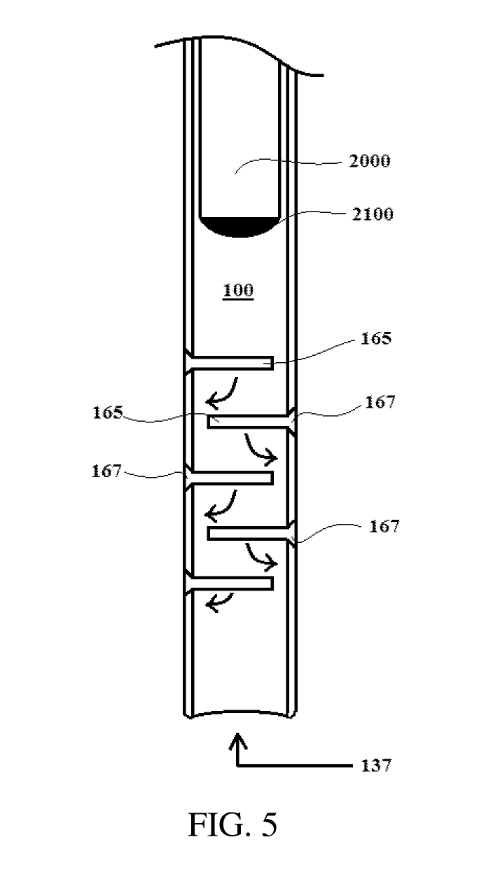

Referring specifically to FIG. 1, there is shown an external, perspective view of a sterile, disposable endoscope cleaning apparatus 1000, particularly well-suited to cleaning the distal lens 2100 of a laparoscope 2000 of FIG. 4, according to an exemplary aspect of the present invention. Cleaning apparatus 1000 comprises hollow tube 100 having a proximal end 110, wherein proximal end 110 includes stop means/anchoring portion 120 for preventing apparatus 1000 from sliding through a cannula 3000 (FIG. 4); and a distal end portion 130, wherein distal end portion 130 includes cleaning portion 140, distal end portion terminating distally in an openable distal end 135.

Elongated hollow tube 100 is preferably made of a thin walled, flexible, sterilizable, biocompatible material, not limited to such as plastic, stainless steel, cobalt chrome, or titanium. Hollow tube 100 can also be made of a nano-composite material which can be shaped into a thin walled tube. Preferably, the wall thickness of hollow tube 100 is around 0.004 inches, but it may be of any desired thickness. The overall length of hollow tube 100 is longer than the length of cannula 3000 (FIG. 4), but it can nevertheless have varying lengths depending on the particular surgery to be performed and the ancillary instruments to be used therewith.

Referring now to FIG. 4, the inner diameter of hollow tube 100 can be sized to accommodate any scope 2000, such as exemplary laparoscope 2000 shown in FIG. 4. Preferably, the inner diameter of hollow tube 100 is sized to accommodate robotic laparoscopes like those found in the Da Vinci™ surgical system. Also as shown, the outer diameter of hollow tube 100 fits within the inner diameter of cannula 3000. For each standard cannula size, there would ideally be a cleaning apparatus 1000 designed to fit smoothly and slidably into the cannula. In some circumstances, particularly if the laparoscope is excessively dirty, it may be beneficial for the user to clean the scope lens outside of the body, under direct visualization. In that case, apparatus 1000 is held in the hand of the user, rather than within a cannula.

Advantageously, hollow tube 100 of the present invention is suitable for any desired type of scope or trocar and any laparoscope can fit within the hollow tube 100. Surgeons may therefore use equipment of their own choosing. Accordingly, when a surgeon acquires the cleaning apparatus 1000, there will be no need to replace any previously owned set of scope type surgical equipment.

Referring to FIG. 4, it shows that stop means 120 at proximal end 110 of hollow tube 100, in one exemplary embodiment, comprise a broad peripheral flange 120, larger in radial diameter than an outer diameter of hollow tube 100 and larger than an outer diameter of cannula 3000 to be used therewith. The largest diameter of anchoring portion 120 is preferably larger than 12 millimeters and more preferably is between 14-20 millimeters.

Anchoring portion 120 may comprise any suitable shape for preventing loss of hollow tube 100 through the cannula 3000. In the exemplary embodiment of FIGS. 1 and 4, anchoring portion 120 is generally oval in shape, including opposing radial flats or surface areas 126 and 127 which prevent cleaning apparatus 1000 from rolling off any surgical surface (not shown) onto which it may be placed. An upper portion of anchoring portion 120 should further include a funnel shaped entry port 131 leading into central bore 128 for guiding instruments into the central bore 128 of hollow tube 100.

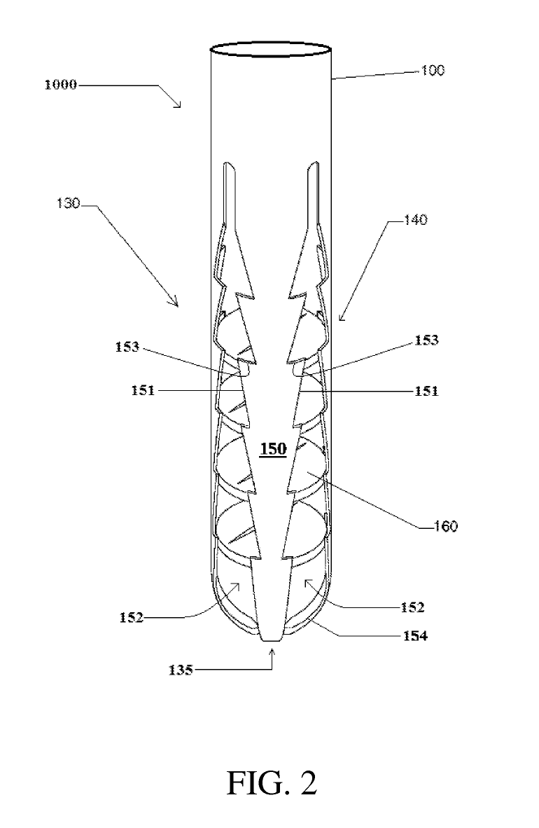

Turning back now to FIG. 2, cleaning portion 140 of cleaning apparatus 1000 is formed at and within the distal end portion 130 of hollow tube 100. Cleaning portion 140 may be configured with any of a variety of suitable shapes and surface patterns which enhance its cleaning surface area; thus the exemplary embodiment illustrated herein should not be construed as limiting. The cleaning portion 140 may further incorporate cleaning members in the form of absorbent cleaning means or pads 160 to contain a cleaning solution facilitating distal scope lens cleaning or to carry a coating for application onto a distal scope lens to improve visualization. In a preferred embodiment, cleaning portion 140 comprises two structures: receiving members 150 and cleaning members 160.

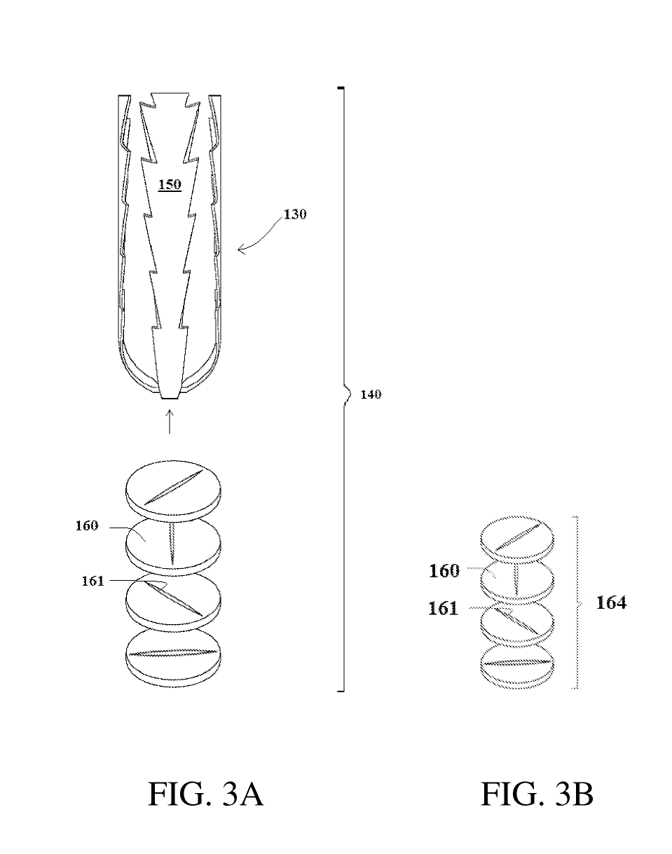

As shown best in FIG. 4, receiving members 150 are formed between and defined by adjacent cutouts 152 formed in distal end portion 130 which extend upwardly from the inwardly a curved distal end 135 of the tube 100. In the exemplary embodiment illustrated, each receiving member 150 is in configured to form a plurality of downwardly directed arrows or spearheads 150 which, in a preferred embodiment taper toward curved distal end 135 of hollow tube 100, with the cleaning members 160 held in place in the area of each prong by attachment through gluing, solvent bonding, or press fit to hold the cleaning members or pads 160 in place as the distal lens 2100 of laparoscope 2000 passes through an opening or slit 161 in each cleaning member 160. Since the receiving members 150 curve inwardly toward distal end 135 and are flexible, when the distal lens 2100 of a laparoscope 2000 passes along the cleaning portion 140 of the hollow tube 100, the inwardly curved ends 154 FIG. 2 of receiving members 150 flex outwardly, with outward flexing of the curved ends 154 FIG. 2 of the receiving members 150 stretching the elastic cleaning members 160 tightly across the interior expanse of the hollow tube 100. As best shown in FIG. 3, each cleaning member 160 incorporates a slit 161, which is preferably created therein at an off center position. In the exemplary embodiment, more than one cleaning member 160 is used, and it is preferred that four or more cleaning members 160 are used and are preferably arranged such that the slits 161 are staggered to enhance the cleaning of laparoscope 2000 of FIG. 4.

FIG. 2 provides an enlarged view of cleaning portion 140 of cleaning apparatus 1000 and shows cleaning members 160 held in place and engaged to the receiving members 150, the cleaning members 160 being held to the receiving members 150 the preferred or any other suitable bonding method.

In the exemplary embodiment, receiving members 150 having two mirror image angled sides 151 terminating in two points 153. The terminating points 153 create barbs 153 to which the cleaning members 160 are fixedly attached through application of suitable bonding as described above. Each circumferential group of cooperating barbs 153 at particular levels along the longitudinal axis of hollow tube 100 creates holding or engagement means for one cleaning member 160, the distance between each circumferential group of barbs 153 determining the spacing between cleaning members 160 within hollow tube 100.

In the exemplary embodiment of cleaning apparatus 1000 shown in FIG. 3, slits 161 of cleaning members 160 are illustrated as extending substantially across the diameter of the cleaning members 160. As an endoscope, such as a laparoscope 2000 FIG. 4 passes through the slits 161, a distal lens 2100 thereof is wiped across the lint-free material of the elastic cleaning member 160, and blood and other material collected on the surface of the distal lens 2100 are wiped from the surface of the lens 2100 via such contact. The number of cleaning members 160 can be varied to improve the cleaning effect of apparatus 1000. Cleaning members 160 are preferably made of any lint-free, cottonoid, fibrous, absorbent cleaning material, and elastic material capable of absorbing blood or other fluids. The cleaning members 160 can be made of one or more layers of elastic, sterilizable, absorbent cleaning material, and may be provided with a firm core or base made of non-absorbent material.

In a different embodiment, pads or flaps are used in a staggered orientation to clean off the endoscopic lens. The flaps may be made of but not limited to rubbers, plastics, foam like materials, cloths, microfibers or absorbent materials. The flaps are placed in a nested position, giving way to the path of the endoscope. The direction of the flaps expands downward and outward of the path of the endoscope. This type of design is not limited to but ideal for having an endoscopic cleaner inside an area where expansion of the distal end portion 130 is not possible. As the endoscope goes thru the flaps the distal lens is simultaneously cleaned. When the endoscope is retrieved the flaps return to their normal position. The flaps may be held in place thru a tapered recessed area that holds the top head of the flaps firmly in place. The flaps may also be in a hinged type configuration. The flaps may also be staggered or opposing in configuration. The benefits of using the nested flat configuration is that the flaps may also expand downward and away allowing the endo scope to pass thru to the distal end without expanding the distal end.

FIG. 3A provides an enlarged view of cleaning members 160. Cleaning members 160 may be joined together as one continuous strip or can be composed of individual pads. In FIG. 3A, the most proximal cleaning member 160 shows slit 161 through which at least the distal lens 2100 of laparoscope 2000 of FIG. 4 passes. In its relaxed state the slit 161 of each cleaning member 160 is almost closed so that both side surfaces 160 of the slit 161 are touching. Cleaning members 160 are preferably comprised of a suitable elastic lint-free material so as to stretch across the diameter of the tube 100, i.e., cleaning members 160 are positioned in a staggered of center manner with respect with the slits 161.

In one preferred embodiment shown in FIG. 3A, the slits 161 of cleaning members 160 should not be all centered along the length of the distal end portion 130. Most slits 161 should be off center to enhance cleaning of substantially the entire surface of the distal lens 2100 of FIG. 4 of the laparoscope 2000, with different cleaning members 160 cleaning different surface areas of the distal lens 2100. The cleaning members 160 are preferably mounted perpendicular to the axis of cannula 3000 of FIG. 4, though this should not be construed as limiting. Each cleaning member 160 is also relatively thin to reduce any frictional forces created as the shaft 2200 of laparoscope 2000 passes through the plurality of cleaning members 160. The plurality of cleaning members 160 is oriented in a manner which takes into consideration the standard angles of distal lenses of substantially all endoscope manufacturers.

Referring to FIG. 4, where there is shown a cross sectional view of an exemplary embodiment of the present invention, in use, laparoscopic cleaner 1000 is inserted into lumen 3100 of cannula 3000 with anchoring portion 120 preventing the laparoscopic cleaning apparatus 1000 from being pushed into a surgical site. FIG. 4 also illustrates a Trocar valve 200.

Once laparoscope cleaning apparatus 1000 is positioned in cannula 3000 in a manner where distal end portion 130 thereof extends outwardly of the cannula 3000, laparoscope 2000 is inserted into the central bore 128 of the hollow tube 100 of cleaning apparatus 1000 via funnel shaped entry port 131. Laparoscope 2000 is cleaned by passage through the slits 161 of cleaning members 160 in distal end portion 130 of hollow tube 100. The lens 2100 of laparoscope 2000 should exit distal end 135 of hollow tube 100, upon opening the distal end 135, into a patient's abdomen or other cavity. The cleaning portion 140 of laparoscopic cleaning apparatus 1000 preferably extends beyond the distal end of cannula 3000 to allow for outward motion or flexing of the curved distal ends 154 of the receiving members 150.

The above-described embodiments are merely exemplary illustrations of implementations set forth for a clear understanding of the principles of the invention. Many variations, combinations, modifications, or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

What is claimed is:

1. A disposable apparatus for cleaning a distal lens of an endoscope, said apparatus comprising:
a hollow tube, said hollow tube including a longitudinal axis and an inner diameter configured to slidably receive a distal end of an cooperating endoscope; said hollow tube further having an outer diameter configured to be slidably received within a corresponding cannula, said cleaning apparatus further including a proximal open end and circumferential means for preventing the proximal end of the hollow tube from entering the cannula;
said hollow tube further including a plurality of elastic cleaning members disposed across the longitudinal axis of said hollow tube and within the distal portion of said hollow tube; said plurality of cleaning members configured to contact and wipe at least a portion of the distal lens of an endoscope as the scope translates through said hollow tube along the longitudinal axis.

2. The apparatus of claim 1 being sterile.

3. The apparatus of claim 1 wherein the proximal opening is funnel shaped for guiding the endoscope into the hollow tube.

4. The apparatus of claim 1 wherein the proximal end of the tube includes a circumferential peripheral flange of greater diameter than a diameter of a cannula with which it is to be used.

5. The apparatus of claim 1 wherein the hollow tube includes a distal end portion within which the cleaning members are suitably fixed.

6. The apparatus of claim 5 wherein the distal end portion of the tube includes a plurality of cooperating sets of upwardly barbed elements in longitudinal spaced relationship to one another extending about an inner periphery of the hollow tube to each cooperating set of which a cleaning elements are attached.

7. The apparatus of claim 1 wherein each cleaning element comprises a lint-free, elastic, absorbent cleaning material disk having a slit therein.

8. The apparatus of claim 1 wherein each cleaning element comprises a lint-free, elastic material having flaps made of absorbent cleaning material extending downward in a nested configuration disposed to allow passage of an endoscope.

9. The apparatus of claim 1 wherein each of the cleaning elements comprises a disk having an off-center slit therein.

10. The apparatus of claim 1 wherein each of the cleaning elements comprises a disk having a different orientation of the slit.

11. The apparatus of claim 9 wherein the cleaning elements are orientated within the hollow tube in a manner to stagger the position of the slits relative to one another.

12. The apparatus of claim 1 wherein the cleaning elements are fixed in position within the distal end portion of the hollow tube by suitable means.

13. The apparatus of claim 1 wherein the tube is longer than a length of a corresponding cannula.

14. The apparatus of claim 6 wherein the barbed elements are flexible to flex outwardly upon passage of an endoscope therebetween.

15. The apparatus of claim 1 wherein the cleaning members are fixedly engaged to receiving members defined as longitudinal corresponding pluralities of arrow shaped members which are defined between corresponding adjacent cutouts created in the distal end of the hollow tube.

16. The apparatus of claim 14 wherein the receiving members include inwardly curved distal ends.

17. The apparatus of claim 15 wherein the inwardly curved distal ends of the receiving members are outwardly flexible.

18. The apparatus of claim 16 wherein the inwardly curved distal ends of the receiving members are flexed outwardly upon passage of the distal lens of an endoscope therethrough.

19. The apparatus of claim 14 wherein the cleaning members are fixedly engaged to the receiving members through use of glue or solvent bonding.

20. A disposable apparatus for cleaning a distal lens of an endoscope comprising a hollow tube having an open proximal end and an openable distal end and being sized and configured to receive an endoscope therethrough, the apparatus including a plurality of lens cleaning members disposed across a diameter of the hollow tube, the cleaning members each including a slit through which the distal lens passes with the lint-free material of the cleaning members wiping across the lens as it passes therethrough.

21. The apparatus of claim 20 wherein the apparatus is configured for use in the hand of a user.
